(19) United States
(12) Patent Application Publication
(10) Pub. No.: US 2026/0283729 A1
(43) Pub. Date: Sep. 24, 2026

(54) SYSTEM AND METHOD OF OPERATING SURGICAL ROBOTIC SYSTEMS WITH ACCESS PORTS OF VARYING LENGTH

(71) Applicant: Covidien LP, Mansfield, MA (US)

(72) Inventors: Andrew W. Zeccola, Salem, MA (US); Jared N. Farlow, Los Angeles, CA (US); Jaimeen V. Kapadia, Cambridge, MA (US); Paul M. Loschak, Somerville, MA (US); Alok Agrawal, New Haven, CT (US); Gregory A. Dierksen, Arlington, MA (US); Sanjay Jonnavithula, New York, NY (US); Colin H. Murphy, Cambridge, MA (US)

(21) Appl. No.: 19/693,848

(22) Filed: Jun. 1, 2026

Related U.S. Application Data
(62) Division of application No. 18/686,580, filed on Sep. 27, 2022, now Pat. No. 12,642,613.
(60) Provisional application No. 63/248,695, filed on Sep. 27, 2021.

(51) Int. Cl. A61B 34/37; A61B 17/00; A61B 17/34; A61B 34/00; A61B 34/30; A61B 90/00; B25J 13/06; G05B 19/4155; G06F 3/01

(52) U.S. Cl. CPC A61B 34/37; A61B 17/34; A61B 17/3423; A61B 34/25; A61B 34/74; A61B 90/37; B25J 13/06; G05B 19/4155; G06F 3/012; G06F 3/013; A61B 2017/00216; A61B 34/30; A61B 2034/742; A61B 2090/062; G05B 2219/40269

(57) ABSTRACT

A surgical robotic system is configured to enable user confirmation of whether one or more access ports are standard or longer length (e.g., bariatric type access ports). The system includes a surgeon console having one or more hand controllers and a head tracking device configured to detect surgeon's attempt to actuate an end effector of an instrument disposed within the access port. The system prevents actuation of the end effector until the insertion depth of the instrument is sufficient or the user confirms that the access port is of standard length, e.g., less than a preset length threshold.

### CROSS-REFERENCE TO RELATED APPLICATION

The present application is a divisional of U.S. application Ser. No. 18/686,580, filed Feb. 26, 2024, which claims priority to U.S. National Stage Application filed under 35 U.S.C. § 371(a) of International Patent Application No. PCT/US 2022/044822, filed Sep. 27, 2022, which claims the benefit of and priority to U.S. Provisional Application No. 63/248,695, filed on Sep. 27, 2021, the entire disclosure of which is incorporated by reference herein in its entirety.

### BACKGROUND

Surgical robotic systems are currently being used in minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body.

Certain surgical robotic systems do not support automatic identification of a type of port/trocar that is attached to each arm, so the systems are not aware of port properties, e.g., length.

### SUMMARY

The present disclosure provides a surgical robotic system configured to operate endoscopic instruments inserted through an access port and to operate with access ports of varying lengths. In order to prevent damage to the instrument or the access port, the system prevents actuation of an end effector of the instrument while the end effector is within the access port (e.g., tubular portion). End effector actuation is enabled after a first proximal joint of the end effector is inserted into the patient past a threshold depth. This is done to prevent the instrument from being damaged as well as to prevent damaging the access port. Similarly, during instrument extraction, the system also prevents the user from pulling the instrument through the access port while the instrument is actuated (e.g., the end articulated) to prevent the instrument and the port from being damaged.

In surgery, access ports may have varying lengths. For example, bariatric ports, may be longer than the standard-length ports. Long ports feature the same parts as standard-length ports. The long ports use the same types of port seals and attach to the robot arm by the same port latch. The remote center of motion (RCM) of various length ports is also the same distance from the robot arm. A difference between long ports and standard ports is that the long ports typically feature a few additional centimeters of port length below the RCM (i.e., the portion that extends into the patient). The position of the RCM for the port cannot be changed and thus, the minimum insertion depth before which the instrument clears the port is larger for the bariatric port. This may be problematic for a surgical robotic system that cannot automatically detect the type and/or length of the access port that is installed.

The surgical robotic system according to the present disclosure treats all access ports as bariatric ports (i.e., longer ports) and prevents the user from articulating or otherwise actuating the end effector of the instrument until the instrument is inserted to a depth sufficient to clear the longer port. The system is configured to determine the insertion depth of the instrument and whether the user is controlling the instrument (e.g., moving or actuating the end effector). If the surgeon tries actuating the end effector while the end effector is disposed at an insertion depth between the standard-length port and the longer port, the system outputs an alert on a graphical user interface (GUI) requesting confirmation that a standard-length port is being used and that the instrument has cleared a distal end of the access port. Once the surgeon confirms that the standard-length port is being used the system stores this setting until the access port is undocked from a holder.

According to one aspect of the present disclosure, a surgical robotic system is disclosed. The surgical robotic system includes a robotic arm configured to support an access port and an instrument having an end effector inserted into the access port. The system also includes a surgeon console configured to detect user activity. The system further includes a controller configured to: monitor an insertion distance of the instrument within the access port, analyze the user activity, and prevent actuation of the end effector in response to the user activity while the insertion distance is less than a threshold distance.

Implementations of the above embodiment may include one or more of the following features. According to one aspect of the above embodiment, the surgeon console also may include a hand controller having a gimbal assembly and a finger sensor. The user activity may include contacting the finger sensor and/or rotating the hand controller about the gimbal assembly. The surgeon console may also include a head tracking device configured to detect head position and/or eye direction. The user activity may further include head and/or eye pointing toward the surgeon console.

The surgical robotic system may also include a display. The controller may be further configured to output a confirmation query on the display in response to detection of the user activity and the insertion distance being less than the threshold distance. The display may be a touchscreen, and a response to the confirmation query may include touching the touchscreen. The head tracking device may be configured to detect a head nod as the response to the confirmation query. The controller may be further configured to enable actuation of the end effector in response to a confirmation that a length of the access port is less than the threshold distance.

According to another aspect of the present disclosure, a method for controlling a surgical robotic system is disclosed. The method may include monitoring user activity at a surgeon console; monitoring an insertion distance of an instrument having an end effector into an access port, the instrument and the access port supported by a robotic arm. The method may also include detecting the user activity. The method may further include preventing actuation of the end effector in response to the user activity while the insertion distance is less than a threshold distance.

Implementations of the above embodiment may include one or more of the following features. According to one aspect of the above embodiment, the surgeon console may include a hand controller having a gimbal assembly and a finger sensor. Detecting the user activity may include detecting at least one of contacting the finger sensor or rotating the hand controller about the gimbal assembly. Detecting the user activity may include tracking at least one head position or eye direction at a head tracking device of the surgeon console. Tracking the head position and/or the eye direction may include detecting at least one of head or eye pointing toward the surgeon console. The method may further include displaying a graphical user interface on a display. The method may further include outputting a confirmation query on the display in response to detection of the user activity and the insertion distance being less than the threshold distance. The method may also include receiving a response to the confirmation query at a touchscreen of the display. The method may further include detecting a head nod as the response to the confirmation query at the head tracking device. The method may also include enabling actuation of the end effector at a controller in response to a confirmation that a length of the access port is less than the threshold distance.

According to yet another aspect of the present disclosure, a surgical robotic system is disclosed, which includes a robotic arm configured to support an access port and an instrument having an end effector inserted into the access port. The system also includes a surgeon console having a hand controller configured to control at least one of the robotic arm or the instrument and a display. The system further includes a controller configured to output a first prompt on the display, the first prompt requesting user identification a property of the access port and confirm the property of the access port. The controller is further configured to output a second prompt on the display, the second prompt requesting user affirmation of the property of the access port based on a confirmation of the property of the access port.

Implementations of the above embodiment may include one or more of the following features. According to one aspect of the above embodiment, the property of the access port may be length. The first prompt may include a query requesting user selection of a type of the access port, the type being either standard or long. In confirming the property of the access port, the controller may be further configured to determine whether the instrument is operating outside a virtual boundary. In determining whether the instrument is operating outside a virtual boundary, the controller may be further configured to count a number of operations outside the virtual boundary and duration of the operations outside the virtual boundary.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms according to an embodiment of the present disclosure;

FIG. 2 is a perspective view of a surgical robotic arm of the surgical robotic system of FIG. 1 according to an embodiment of the present disclosure;

FIG. 3 is a perspective view of a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a side view of access ports of different lengths according to the present disclosure;

FIG. 6 is an enlarged perspective view of a hand controller of the surgeon console of FIG. 1;

FIG. 7 is a flow chart of a method for controlling the surgical robotic system of FIG. 1 according to one embodiment of the present disclosure;

FIG. 8 is a flow chart of a method for controlling the surgical robotic system of FIG. 1 according to another embodiment of the present disclosure; and

FIG. 9 is a plan schematic view of movable carts of FIG. 1 positioned about a surgical table according to an aspect of the present disclosure.

### DETAILED DESCRIPTION

Embodiments of the presently disclosed surgical robotic system are described in detail with reference to the drawings, in which like reference numerals designate identical or corresponding elements in each of the several views. As used herein the term “distal” refers to the portion of the surgical robotic system and/or the surgical instrument coupled thereto that is closer to the patient, while the term “proximal” refers to the portion that is farther from the patient.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, a personal computer, or a server system.

As will be described in detail below, the present disclosure is directed to a surgical robotic system, which includes a surgeon console, a control tower, and one or more movable carts having a surgical robotic arm coupled to a setup arm. The surgeon console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

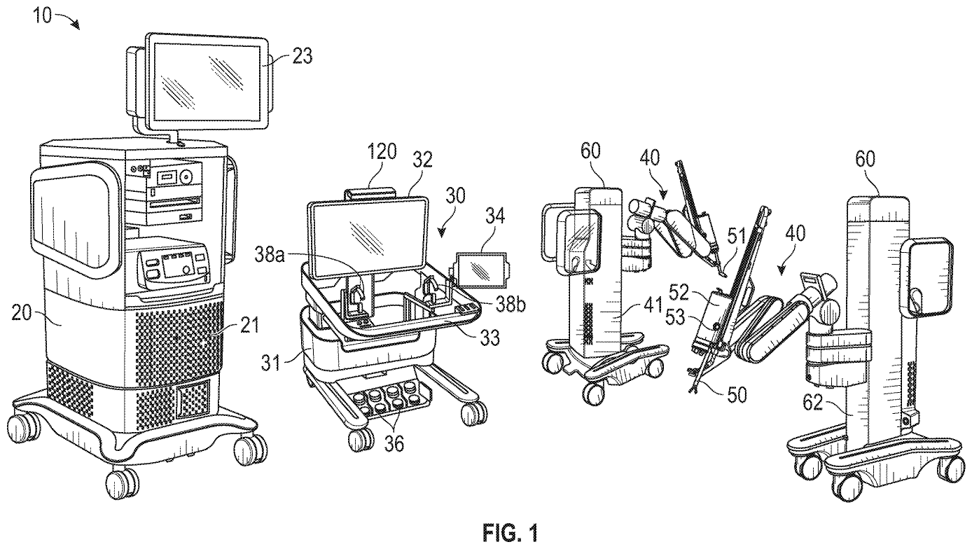

With reference to FIG. 1, a surgical robotic system 10 includes a control tower 20, which is connected to all of the components of the surgical robotic system 10 including a surgeon console 30 and one or more robotic arms 40. Each of the robotic arms 40 includes a surgical instrument 50 removably coupled thereto. Each of the robotic arms 40 is also coupled to a movable cart 60.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include a camera 51 configured to capture video of the surgical site. The surgeon console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgeon console 30 also includes a plurality of user interface devices, such as foot pedals 36 and a pair of hand controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the hand controllers 38a and 38b.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

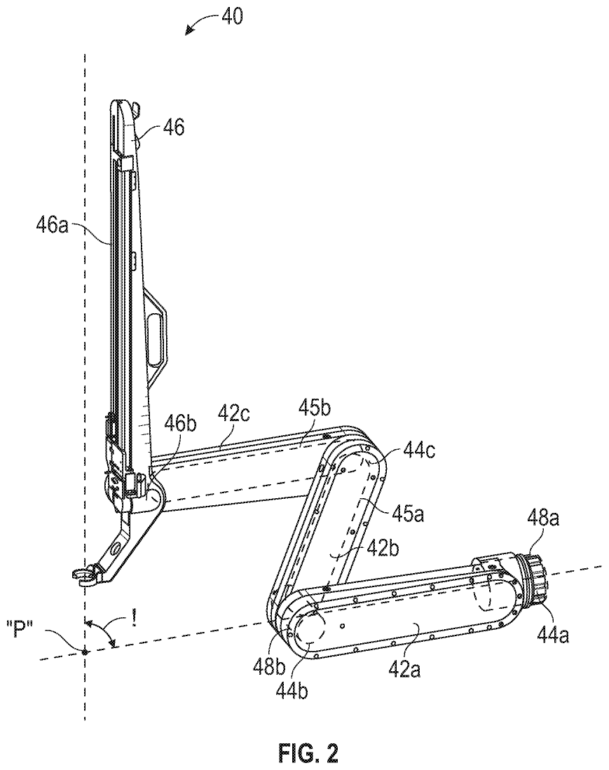

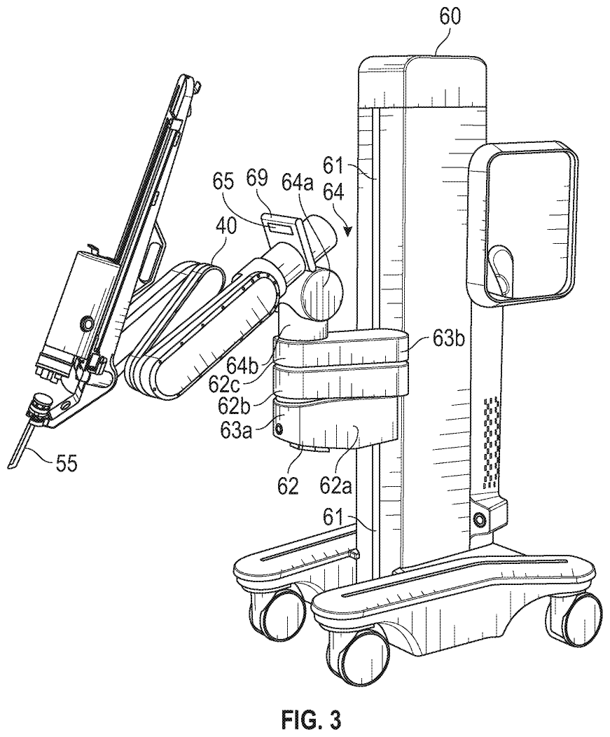

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the robotic arm 40 also includes a holder 46 defining a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic port 55 (FIG. 3) held by the holder 46.

The robotic arm 40 also includes a plurality of manual override buttons 53 (FIG. 1) disposed on the IDU 52 and the setup arm 62, which may be used in a manual mode. The user may press one or more of the buttons 53 to move the component associated with the button 53.

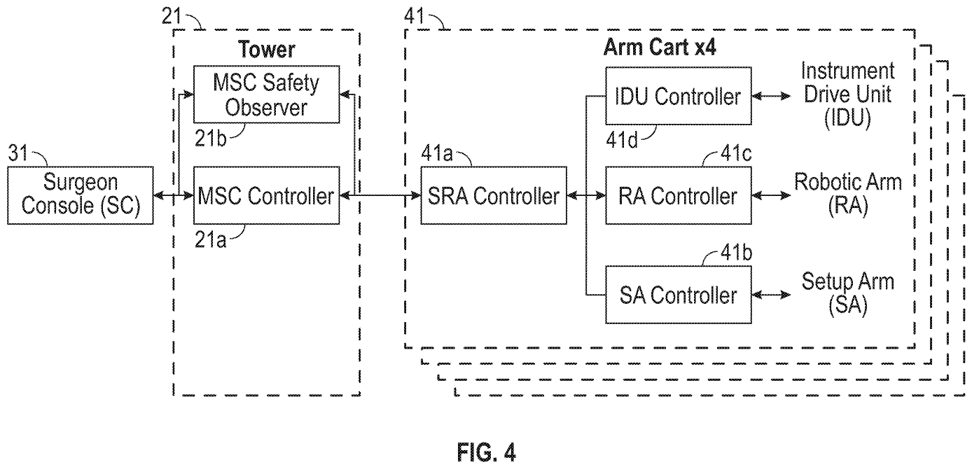

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the hand controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the hand controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the hand controller controlling the robotic arm 40, e.g., the hand controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the hand controller 38a may be embodied as a coordinate position and role-pitch-yaw (“RPY”) orientation relative to a coordinate reference frame, which is fixed to the surgeon console 30. The desired pose of the instrument 50 is relative to a fixed frame on the robotic arm 40. The pose of the hand controller 38a is then scaled by a scaling function executed by the controller 21a. In embodiments, the coordinate position is scaled down and the orientation is scaled up by the scaling function. In addition, the controller 21a also executes a clutching function, which disengages the hand controller 38a from the robotic arm 40. In particular, the controller 21a stops transmitting movement commands from the hand controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the hand controller 38a or 38b is then passed by an inverse kinematics function executed by the controller 21a. The inverse kinematics function calculates angles for the joints 44a, 44b, 44c of the robotic arm 40 that achieve the scaled and adjusted pose input by the hand controller 38a or 38b. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

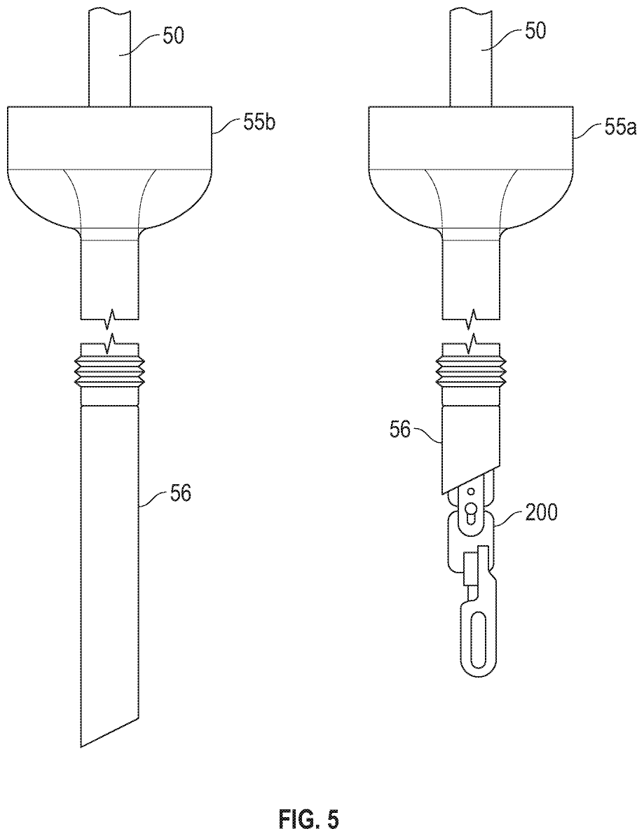

With reference to FIG. 5, during surgery, ports of different lengths may be used, in particular, long ports 55b may be used for bariatric surgery, which are longer than standard ports 55a. Use of longer ports affects the functionality of the system 10 during instrument exchange and position control of the instrument 50 inside the port 55. More specifically, instrument exchange requires the wristed instrument to be straightened and closed during extraction before the first proximal joint reaches the bottom of the port.

During use of the instrument 50, the instrument 50 is inserted into a longitudinal tube 56 of the port 55 by advancing the instrument 50 and the IDU 52 along the sliding mechanism 46a. The instrument 50, and in particular an end effector 200 is advanced to a desired depth. The distance to which the instrument 50, and in particular, the end effector 200 has been advanced is continuously tracked by the IDU controller 41d and other controllers (e.g., controller 21a).

The surgical robotic system 10 is configured to determine whether the user of the surgeon console 30 is attempting to control the instrument 50 and/or end effector 200. Attempts to control the instrument 50 include inputting activation commands through the hand controllers 38a and 38b to actuate the end effector 200 as well as whether the surgeon is facing or engaging with the surgeon console 30. The surgical robotic system 10 utilizes the following hardware of the surgeon console 30 to determine user's intent to control the instrument 50.

With reference to FIG. 1, the surgeon console 30 includes a head tracking device 120, which may include one or more cameras, illuminating devices, such as IR projectors and the like. By way of the tracking device 120, the surgeon console 30 is configured to, in real-time or near real-time, identify and track a user's head and user's eyes. Head position and/or eye direction is used by the controller 31a of the surgeon console 30 to determine whether the user is engaged with, or disengaged from, the surgeon console 30.

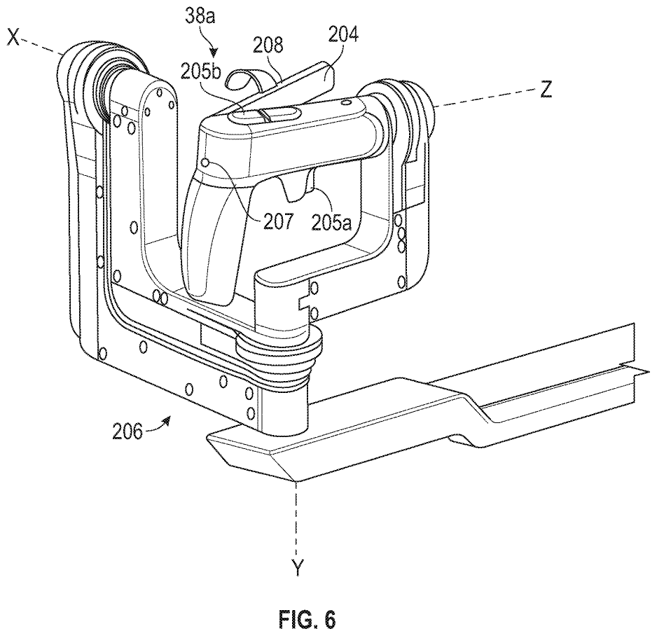

The surgeon console 30 is also configured to track user engagement with the hand controllers 38a and 38b. FIG. 6 shows the right-hand controller 38b, which is a mirror copy of the left-hand controller 38b. Each of the hand controllers 38a and 38b includes a paddle 208 configured controlling actuation of the end effector, e.g., opening and closing jaws of the end effector 200. The paddle 208 may include a finger sensor 204 configured to detect presence or movement of a finger, such as touch sensors, capacitive sensors, optical sensors, and the like. In embodiments, the finger sensor 204 may be disposed on any portion of the hand controllers 38a and 38b. Each of the hand controllers 38a and 38b may also include a trigger 205a and one or more buttons 205b for activating various functions of the instrument 50. In addition, each of the hand controllers 38a and 38b may include a gimbal assembly 206 allowing for movement and rotation of the hand controllers 38a and 38b about three axes (x, y, z). The hand controllers 38a and 38b may also include an infrared proximity sensor 207 configured to detect hand contact with a grip of the hand controllers 38a and 38b. Details of the hand controllers 38a and 38b are provided in U.S. Patent Publication No. 2020/0315729, titled “Control arm assemblies for robotic surgical systems” filed on Nov. 30, 2018, the entire contents of which are incorporated by reference herein.

The controller 31a of the surgeon console 30 monitors user interactions with the hand controllers 38a and 38b and controls the instrument(s) 50 in response to user inputs. In addition, the controller 31a also monitors individual or a new velocity of each joint of the gimbal assembly 206. The controller 31a further monitors displacement of each of the joint of the gimbal assembly 206 and/or net displacement of the gimbal assembly 206. Furthermore, the controller 31a may also monitor the input angle and/or velocity of the paddle 208.

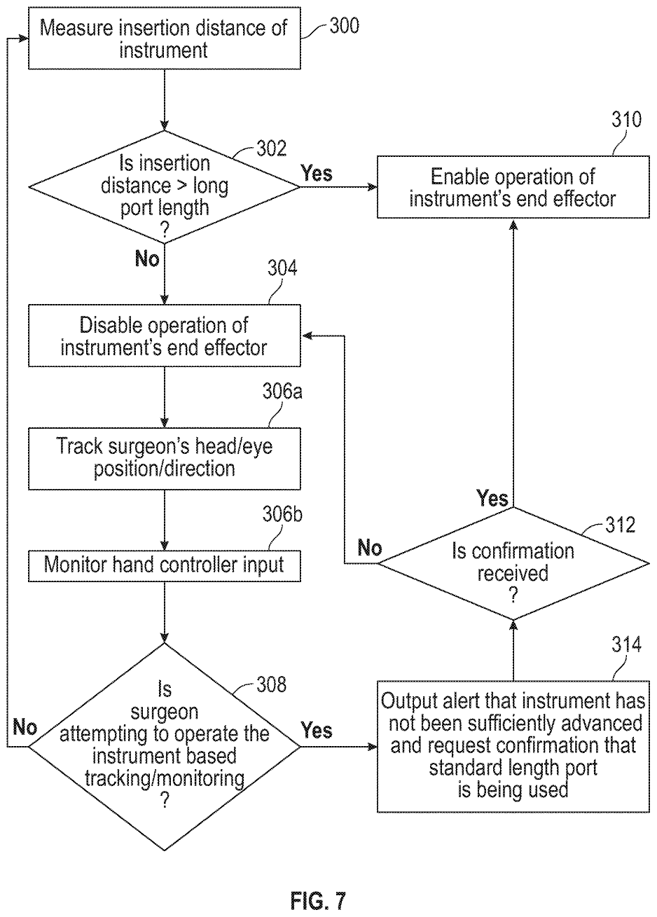

With reference to FIG. 7, a method for verifying length of the access port 55 includes at step 300 determining (i.e., measuring) the depth of insertion of the instrument 50 into the access port 55. Insertion distance tracking may be determined using encoders or other sensors tracking the sliding mechanism 46a, which is configured to move the IDU 52 along the second longitudinal axis defined by the holder 46. The distance threshold is set to be longer than the length of the long port 55b to ensure safe actuation of the instrument 50 and/or the end effector 200. At step 302, the measured insertion distance is compared a threshold, which corresponds to a length of the long port 55b. If the insertion distance or the depth of the instrument 50 is longer than the length of the long port 55b then the controller 21a allows actuation of the instrument 50 and/or the end effector 200 at step 310.

At step 304, if the insertion distance or the depth of the instrument 50 is less than the length of the long port 55b then the controller 21a prevents actuation of the instrument 50 and/or the end effector 200 by disabling actuation of the instrument 50. Disabling of the instrument 50 may be accomplished by ignoring user input commands from the surgeon console 30 at the IDU 52. While actuation of the instrument 50 is disabled, the controller 31a of the surgeon console 30 monitors user activity as step 306 with respect to the surgeon console 30. User activity includes any interaction by the surgeon with the surgeon console 30. In particular, at step 306a, the controller 31a tracks surgeon's head and eye movement and direction to determine whether the surgeon is looking at the display 32. If the surgeon is looking at the display 32, then it is determined that the surgeon is intending to control the instruments 50.

Additionally or alternatively, at step 306b, the controller 31a may also monitor surgeon's engagement with the hand controllers 38a and 38b by monitoring finger sensors 204, proximity sensors 207, movement of the gimbal assembly 206, pressing trigger 205a or buttons 205b, and/or movement of the paddles 208. If user input of a predetermined type and/or magnitude is detected at step 308, while the insertion distance of the instrument 50 is less than the threshold distance, then the controller 21a outputs an alert at step 314 to the surgeon and/or other staff indicating that the instrument 50 has not been sufficiently advanced and requesting confirmation at step 312 that the standard-length port 55a is being used.

The alert and confirmation query may be output on any of the displays 23, 32, and 34 of the control tower 20 and the surgeon console 30, respectively. Confirmation may be received by touching a corresponding button, e.g., “YES”, displayed on any of the displays 23, 32, and 34. The surgeon may also input the confirmation by shaking the head affirmatively (e.g., a vertical nod), which can be tracked by the head tracking device 120. In embodiments, the surgeon may input confirmation by touching the finger sensors 204 using any suitable gesture, such as a double tap, pressing the trigger 205a and/or buttons 205b, etc. Furthermore, the surgeon may press one of the foot pedals 36 of the surgeon console 30 to confirm the access port length. In response to confirmation, the controller 21a may store the confirmation and modify any parameters associated with the confirmation, such as the threshold distance. This would prevent the surgeon reentering confirmation on subsequent uses (i.e., insertion) of the instrument 50.

Once confirmation is received, then the controller 21a enables actuation of the instrument 50 and/or the end effector 200 at step 310 in response to the surgeon's inputs. The method of FIG. 7 may be carried out for each of the access ports 55 held by each of the robotic arms 40 until all of the instruments 50 being used have been activated. In embodiments, another confirmation may be used to apply the same port selection to each of the robotic arms 40 to minimize the number of prompts being displayed.

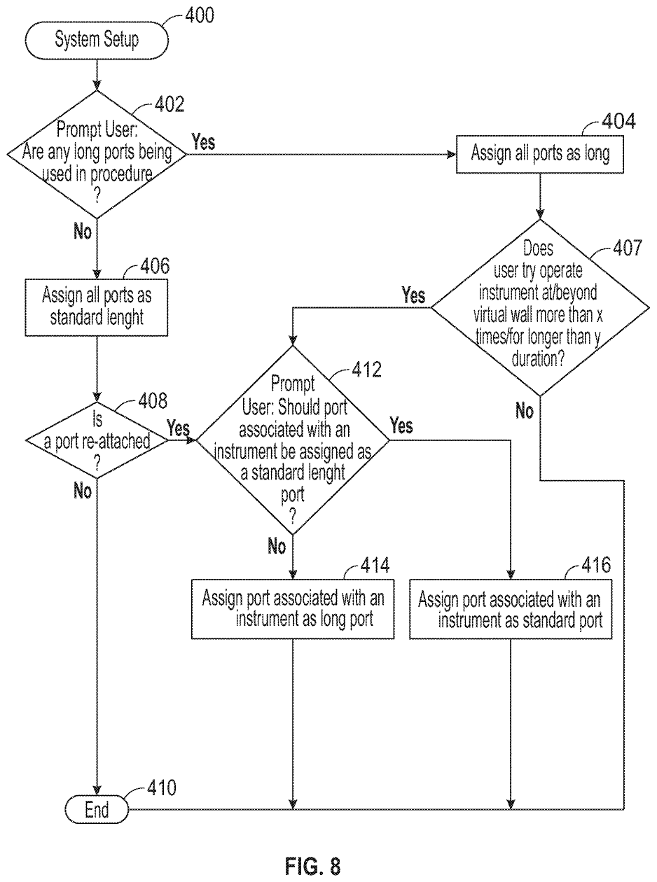

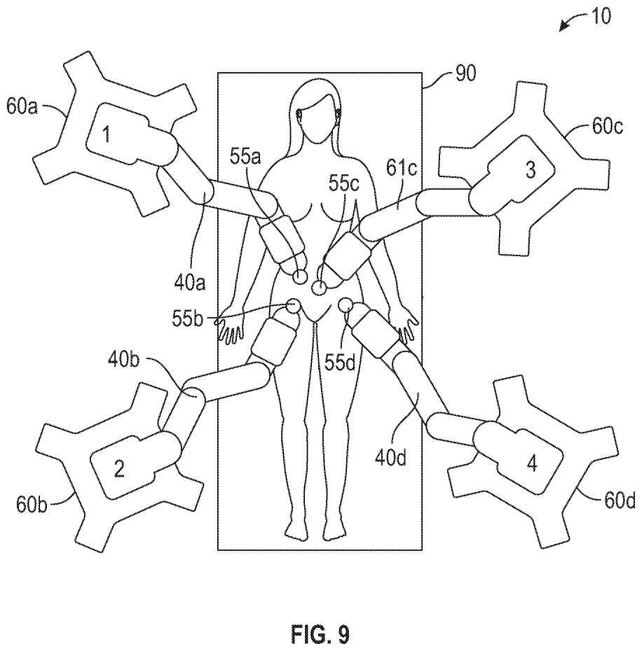

With reference to FIG. 8, another method for verifying length of the access port 55 includes at step 400 setting up the system as shown in FIG. 9. The methods of FIGS. 7 and 8 may be implemented as software instructions executable by any of the controllers of the system 10 (e.g., controllers 21a, 31, 41a, etc.).

In embodiments, the surgical robotic system 10 is setup around a surgical table 90. (See FIG. 9.) The system 10 includes movable carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placement is determined, the access ports 55a-d are inserted into the patient, and carts 60a-d are positioned to insert instruments 50 and the endoscopic camera 51 into corresponding ports 55a-d.

As described above, different types of access ports 55a-d may be used. In particular, access ports having different dimensions (e.g., diameter and lengths) may be used. The access port 55a may be a standard length access port having a length of from about 100 mm to about 200 mm, whereas the access port 55b may be a long length port, having a length of 200 mm or above. Port diameter may be different for different ports, and may be from about 5 mm to about 15 mm.

As step 402, the system 10 provides a prompt to the user (e.g., via a GUI on the first display 32) asking whether there are any long ports that are being used. The prompt may be a pop-up query with “YES” and “NO” responses. If the response is “YES”, then the system 10 sets all of the access ports 55a-d as long length ports at step 404. If the response is “NO” then the system 10 sets all of the access ports 55a-d as standard length ports at step 406. In embodiments, the system 10 may provide individual prompts for each of the access ports 55a-d and receive responses for each prompt and access port.

During operation of the robotic arms 40a-d and the instruments 50 in response to user inputs at surgeon console 30, the system 10 monitors whether the user is attempting to operate the instrument 50 beyond a virtual boundary of a standard length access port. The system 10 may store in memory or calculate for different types of access ports 55a-d a virtual boundary, e.g., a first 3D space reachable by the instrument 50 when operated through a standard access port and a second 3D space reachable by the instrument 50 when operated through a long length access port. The second 3D space may be smaller than the first 3D space.

At step 407, the system 10 confirms the user's response that all access ports are of long lengths. The system 10 monitors user's operation of the instrument 50. In particular, the system 10 counts the number of times the user attempted to operate the instrument 50 beyond the virtual boundary and whether each of the attempts (i.e., movement and/or position beyond the virtual boundary) exceeded a predetermined time limit. The number of attempts threshold may be 2 or above, and the time threshold may be about 2 seconds or above.

If the system 10 determines that the instrument 50 was operated outside the virtual boundary (i.e., number of attempts longer than the time threshold is larger than the attempt threshold), then the system 10 proceeds to step 412, which prompts the user with a second prompt at step 412 to confirm that the user correctly answered the first prompt at step 402 since the usage of the instrument 50 as monitored by the system 10 contradicts user's initial classification of the access port (i.e., stating the access port is long but the movement confirms that the access port is standard). The prompt asks the user (e.g., via the GUI on the first display 32) whether the access port is a standard or long type access port. The prompt may be a “YES” or “NO” query or a query asking the user to identify the type (e.g., “STANDARD” or “LONG”). Based on the response to the prompt, the system 10 sets the access port type as “standard” at step 414 or as “long” at step 416. If the system 10 determines that the instrument 50 was operated within the virtual boundary, then the process ends at step 410.

During the surgical procedure, the system 10 also continuously monitors detachment and/or reattachment of access ports 55a-d at step 408. If all of the access ports 55a-d that were present during setup of the system 10 remain through the duration of the procedure, then the process ends at step 410. If at any time a port is reattached or a new access port is attached, the system 10 detects the attachment and prompts the user with a second prompt at step 412. The prompt asks the user (e.g., via the GUI on the first display 32) whether the newly attached access port is a standard or long type access port. The prompt may be a “YES” or “NO” query or a query asking the user to identify the type (e.g., “STANDARD” or “LONG”). Based on the response to the prompt, the system 10 sets the access port type as “standard” at step 414 or as “long” at step 416.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

1-20. (canceled)

21. A surgical robotic system comprising:
a robotic arm configured to support an access port and an instrument having an end effector inserted into the access port;
a surgeon console including:
a hand controller configured to control at least one of the robotic arm or the instrument; and
a display;
a controller configured to:
output a first prompt on the display, the first prompt requesting user identification of a property of the access port;
confirm the property of the access port; and
output a second prompt on the display, the second prompt requesting user affirmation of the property of the access port based on a confirmation of the property of the access port.

22. The surgical robotic system according to claim 21, wherein the property of the access port is length.

23. The surgical robotic system according to claim 21, wherein the first prompt includes a query requesting user selection of a type of the access port selected from the group consisting of standard or long access ports.

24. The surgical robotic system according to claim 23, wherein in confirming the property of the access port, the controller is further configured to determine whether the instrument is operating outside a virtual boundary.

25. The surgical robotic system according to claim 24, wherein in determining whether the instrument is operating outside a virtual boundary, the controller is further configured to count a number of operations outside the virtual boundary and a duration of the operations outside the virtual boundary.

26. A method for controlling a surgical robotic system, the method comprising:
outputting a first prompt on a display of a surgeon console, the first prompt requesting user identification of a property of an access port, wherein the access port is configured to receive an instrument supported by a robotic arm and the instrument includes an end effector;
confirming the property of the access port at a controller; and
outputting a second prompt on the display, the second prompt requesting user affirmation of the property of the access port based on the confirmation of the property of the access port.

27. The method according to claim 26, wherein the property of the access port is length.

28. The method according to claim 27, wherein the first prompt includes a query requesting user selection of a type of the access port selected from the group consisting of a first length or a second length, wherein the second length is different from the first length.

29. The method according to claim 28, further comprising assigning a port type to the access port based on a response to the first prompt.

30. The method according to claim 26, wherein the surgeon console includes a hand controller having a gimbal assembly and a finger sensor.

31. The method according to claim 30, wherein confirming the property of the access port includes detecting at least one of contacting the finger sensor or rotating the hand controller about the gimbal assembly.

32. The method according to claim 26, wherein confirming the property of the access port includes determining whether the instrument is operating outside a virtual boundary.

33. The method according to claim 32, wherein determining whether the instrument is operating outside the virtual boundary includes counting a number of operations outside the virtual boundary and a duration of operations outside the virtual boundary.

34. The method according to claim 33, further comprising outputting the second prompt in response to the number of operations outside the virtual boundary exceeding a threshold number or the duration of operations outside the virtual boundary exceeding a threshold duration.

35. The method according to claim 26, further comprising assigning a port type to the access port based on a response to the second prompt.

36. A method for controlling a surgical robotic system, the method comprising:
monitoring operation of an instrument having an end effector relative to a virtual boundary, the instrument and an access port supported by a robotic arm;
determining whether a number of operations of the instrument outside the virtual boundary or a duration of operations of the instrument outside the virtual boundary exceeds a threshold; and
outputting a prompt on a display of a surgeon console requesting user affirmation of a type of the access port in response to the number of operations or the duration exceeding the threshold.

37. The method according to claim 36, wherein the type of the access port is selected from the group consisting of a first length or a second length, wherein the second length is different from the first length.

38. The method according to claim 36, further comprising assigning a port type to the access port based on a response to the prompt.

39. The method according to claim 36, further comprising detecting reattachment of the access port to the robotic arm and outputting the prompt in response to detecting the reattachment.
